(19) United States
(12) Patent Application Publication
(10) Pub. No.: US 2009/0326363 A1
(43) Pub. Date: Dec. 31, 2009

(54) FUSED IMAGE MODALITIES GUIDANCE

(75) Inventors: Lu Li, Grass Valley, CA (US); Ramkrishnan Narayanan, Nevada City, CA (US); Jasjit S. Suri, Roseville, CA (US)

Correspondence Address: MARSH, FISCHMANN & BREYFOGLE LLP, 8055 East Tufts Avenue, Suite 450, Denver, CO, 80237, US

(73) Assignee: EIGEN, LLC, GRASS VALLEY, CA (US)

(21) Appl. No.: 12/434,990

(22) Filed: May 4, 2009

Related U.S. Application Data
(60) Provisional application No. 61/050,118, filed on May 2, 2008.
(60) Provisional application No. 61/148,521, filed on Jan. 30, 2009.

(51) Int. Cl. A61B 5/055; A61B 8/00; G06K 9/00

(52) U.S. Cl. 600/411; 600/437; 382/131

(57) ABSTRACT

An improved system and method (i.e. utility) for registration of medical images is provided. The utility registers a previously obtained volume onto an ultrasound volume during an ultrasound procedure to produce a multimodal image. The multimodal image may be used to guide a medical procedure, In one arrangement, the multimodal image includes MRI and/or MRSI information presented in the framework of a TRUS image during a TRUS procedure.

### CROSS REFERENCE TO RELATED APPLICATION

This application claims benefit of the filing date under 35 U.S.C. §119 to U.S. Provisional Application No. 61/050,118 entitled: “Fused image Modalities Guidance” and having a filing date of May 2, 2008, the entire contents of which are incorporated herein by reference and to U.S. Provisional Application No. 61/148,521 entitled “Method for Fusion Guided Procedure” and having a filing date of Jan. 30, 2009, the entire contents of which are incorporated herein by reference.

### FIELD

The present disclosure pertains to the field of medical imaging, and more particularly to the registration of multiple medical images to allow for improved guidance of medical procedures. In one application, multiple medical images are coregistered into a multimodal image to aid urologists and other medical personnel in finding optimal target sites for biopsy.

### BACKGROUND

Medical imaging, including X-ray, magnetic resonance (MR), computed tomography (CT), ultrasound, and various combinations of these techniques are utilized to provide images of internal patient structure for diagnostic purposes as well as for interventional procedures. One application of medical imaging (e.g., 3-D imaging) is in the detection of prostate cancer. According to the National Cancer Institute (NCI), a man's chance of developing prostate cancer increases drastically from 1 in 10,000 before age 39 to 1 in 45 between 40-59 and 1 in 7 after age 60. The overall probability of developing prostate cancer from birth to death is close to 1 in 6.

Traditionally either elevated Prostate Specific Antigen (PSA) level or Digital Rectal Examination (DRE) has been widely used as a standard for prostate cancer detection For a physician to diagnose prostate cancer, a biopsy of the prostate must be performed. This is done on patients that have either abnormal PSA levels or an irregular digital rectal exam (DRE), or on patients that have had previous negative biopsies but continue to have elevated PSA. Biopsy of the prostate requires that a number of tissue samples (i.e., cores) be obtained from various regions of the prostate. For instance, the prostate may be divided into six regions (i.e., sextant biopsy), apex, mid and base bilaterally, and one representative sample is randomly obtained from each sextant. Such random sampling continues to be the most commonly practiced method although it has received criticism in recent years on its inability to sample regions where there might be significant volumes of malignant tissues resulting in high false negative detection rates. Further using such random sampling it is estimated that the false negative rate is about 30% on the first biopsy.

3-D Transrectal Ultrasound (TRUS) guided prostate biopsy is a commonly used method to test for prostate cancer, mainly due to its ease of use and inexpensiveness. However, it is believed that some malignant cells and cancers can be isochoic in TRUS. That is, differences between malignant cells and surrounding healthy tissue may not be discernable in the ultrasound image. Further, speckle and shadows also make ultrasound images difficult to interpret, and many cancers are often undetected even after saturation biopsies that obtain several (>20) needle samples. Due to the difficulty in ascertaining malignancy in tissues, operators have often resorted to simply increasing the number of biopsy cores, which has been shown to offer no significant improvement. In order to alleviate this difficulty, a cancer atlas was proposed that provided a statistical probability image superposed on the patient's TRUS image to help pick locations that have been shown to harbor carcinoma, e.g. the peripheral zone constitutes about 80% of prostate cancer. While the use of a statistical map offers an improvement over the current standard of care, it is still limited in that it is estimated statistically from a large population of reconstructed and expert annotated 3-D histology specimen. Thus patient specific information is not available in this method.

Although MRI has been around for almost three decades now, its use for cancer diagnosis has been limited. It provides better soft tissue contrast than other image modalities, and cancers are typically seen as lower signal intensities compared to neighboring healthy tissue. More recently use of endorectal coils have provided even higher accuracy in the analysis of seminal vesicle and extracapsular extension, and also the spread of cancer to lymph nodes and bones within the pelvis. Endorectal coils have been shown to provide higher staging accuracy compared to using TRUS. The disadvantage of using MRI however is its poor specificity, i.e. inability to distinguish other abnormalities such as benign prostatic hyperplasia of effects of therapy that also result in decreased signal intensity.

MRSI images offset this disadvantage of MRI images. MRSI images provide essentially a four dimensional image where the first three dimensions correspond to voxel position while the fourth shows metabolite concentrations. The concentration of these metabolites can be used to distinguish cancer from non-cancer tissues. For example, a commonly used measure is the ratio of concentration levels of choline and creatine with citrate, which is abnormal in the case of cancer.

While other imaging procedures such as magnetic resonance imaging (MRI) and magnetic resonance spectroscopy imaging (MRSI) provide improved tissue information, these procedures are both time consuming and difficult to utilize for biopsy/treatment guidance due to the size and physical construction of these imaging devices.

It is against this background that the present invention has been developed.

### SUMMARY OF THE INVENTION

It has been recognized that it would be useful to combine previously obtained information from MRI and MRSI with TRUS to guide a biopsy during a TRUS procedure. However, registration of these modalities with in vivo TRUS must be robust to account for shape variations of the prostate as imaged in different procedures due to patient movement, peristalsis, and deformation induced by the sensor probe. More specifically, fusion of MRI and/or MRSI data with a TRUS volume may require rotating and/or deforming the MRI/MRSI image to superimpose its information onto a TRUS framework.

Accurate segmentation of images is necessary to achieve good results when registering images from different modalities. Segmentation of ultrasound prostate images is a very challenging task due to the relatively poor image qualities. In this regard, segmentation has often required a technician to at least identify an initial boundary of the prostate such that one or more segmentation techniques may be implemented to acquire the actual boundary of the prostate. Alternatively, the prostate could be segmented with MRI offline (prior to biopsy), and could guide the segmentation of the prostate from the TRUS images during biopsy.

According to a first aspect, a system and method (i.e., utility) is provided for use in medical imaging of a prostate of a patient. The utility includes obtaining first surface information (e.g., an MRI surface) from first volume data (e.g., an MRI volume) of a prostate of a patient obtained using a magnetic resonance imaging procedure. An ultrasound volume of the patient's prostate is then obtained, and the first surface information is used to segment the ultrasound image into ultrasound surface information. The first volume data (e.g., MRI volume) is registered to the ultrasound volume, and a multimodal image is generated wherein the first volume data is displayed in the frame of reference of the ultrasound volume. The multimodal image may thus be used to guide a medical procedure such as, for example, biopsy or brachytherapy. In one embodiment, the first volume data may be obtained from stored data.

According to another aspect, the utility may further include obtaining second volume data from a magnetic resonance spectroscopy imaging procedure, wherein the second volume data is co-registered with the first volume data. This second volume may be, for example, MRSI data indicating the likelihood of cancer at each voxel within the prostate volume. For example, concentrations of various metabolites such as creatine, choline, and citrate may be measured during an MRSI procedure. In one embodiment, the ratio of creatine and choline to citrate, which is abnormal in cancerous tissue, may be determined at each voxel to generate a derived volume that includes information about cancer prevalence at each location within the prostate. This volume may in turn be presented as part of a multimodal image used to guide a medical procedure. In another aspect, the utility may include registering a statistical atlas with the ultrasound image and using the statistical atlas to guide the medical procedure.

In one aspect, segmenting the ultrasound volume to produce ultrasound surface information includes using the first surface information to provide an initialized surface. This surface may be allowed to evolve in two dimensions or in three dimensions. If the surface is processed on a slice-by-slice basis, vertices belonging to a first slice may provide initialization inputs to second vertices belonging to a second slice adjacent to the first slice.

According to another aspect, registering the first volume data to the ultrasound volume may include establishing a surface correspondence between the first surface information and the ultrasound surface information and deforming the first surface information to match a boundary on the ultrasound surface information.

According to yet another aspect, registering the first volume data to the ultrasound volume may include warping the first volume data to the ultrasound volume using a nonlinear interpolant that employs surface correspondences for warping.

According to another aspect, a method is provided for use in imaging of a prostate of a patient. The method includes obtaining segmented MRI surface information for a prostate; performing an MRSI procedure on the prostate to obtain a cancer indicator at each of a plurality of voxels; extracting a derived volume from the cancer indicators; performing a transrectal ultrasound (TRUS) procedure on the prostate of the patient, wherein the segmented MRI surface information is used to identify a three-dimensional TRUS surface; elastically warping the segmented surface information and the derived volume onto the three-dimensional TRUS surface to obtain a multimodal image of the prostate; and guiding a medical procedure using information from the multimodal image. The step of elastically warping the segmented surface information and the derived volume onto the TRUS image may be performed during the TRUS procedure itself. This step may be performed on a slice-by-slice basis, may be done in two dimensions or in three dimensions, and/or may include generating a force field on a boundary of the segmented surface information; and propagating the force field through the derived volume to displace a plurality of voxels. Identifying a three-dimensional TRUS surface may include using a force field estimate to deform an initial surface.

In accordance with another aspect, a system is provided use in medical imaging of a prostate of a patient. The system may include a TRUS for obtaining a three-dimensional image of a prostate of a patient; a storage device having stored thereon an MRI volume and a derived volume of the prostate of the patient; and a processor (e.g., a GPU) for registering the MRI volume and the derived volume to the three-dimensional image of the prostate.

### BRIEF DESCRIPTION OF THE DRAWINGS

FIG. 1 shows a cross-sectional view of a trans-rectal ultrasound imaging system as applied to perform prostate imaging.

FIG. 2A illustrates a motorized scan of the TRUS of FIG. 1.

FIG. 2B illustrates two-dimensional images generated by the TRUS of FIG. 2A.

FIG. 2C illustrates a 3-D volume image generated from the two dimensional images of FIG. 2B.

FIGS. 3A-D illustrate a first prostate image, a second prostate image, overlaid prostate images prior to registration and overlaid prostate images after registration, respectively.

FIGS. 4A-C illustrate fusing an MRI image with an ultrasound image to generate a multimodal image.

FIG. 5 illustrates a system for producing a multimodal image during a TRUS procedure.

FIG. 6 illustrates a utility for segmenting a three-dimensional image.

FIG. 7 illustrates a two-dimensional guide processor.

FIG. 8 illustrates a three-dimensional morphing processor.

FIG. 9 illustrates a three-dimensional deforming processor.

FIG. 10 illustrates a utility for identifying a transition zone of a prostate.

FIG. 11 illustrates a prostate image.

### DETAILED DESCRIPTION

Reference will now be made to the accompanying drawings, which assist in illustrating the various pertinent features of the present disclosure. Although the present disclosure is described primarily in conjunction with fusion of MRI/MRSI images with transrectal ultrasound images for prostate imaging and treatment, it should be expressly understood that aspects of the present invention may be applicable to other medical imaging applications. In this regard, the following description is presented for purposes of illustration and description.

Disclosed herein are systems and methods that allow for registering multimodal images to a common frame of reference. In this regard, one or more images may be registered to an ultrasound image during an ultrasound procedure to provide enhanced patient information. Specifically, in the application disclosed herein, previous MRI and MRSI images of a prostate of a patient are registered to a TRUS image of the prostate such that a medical procedure may be performed on a desired location of the prostate.

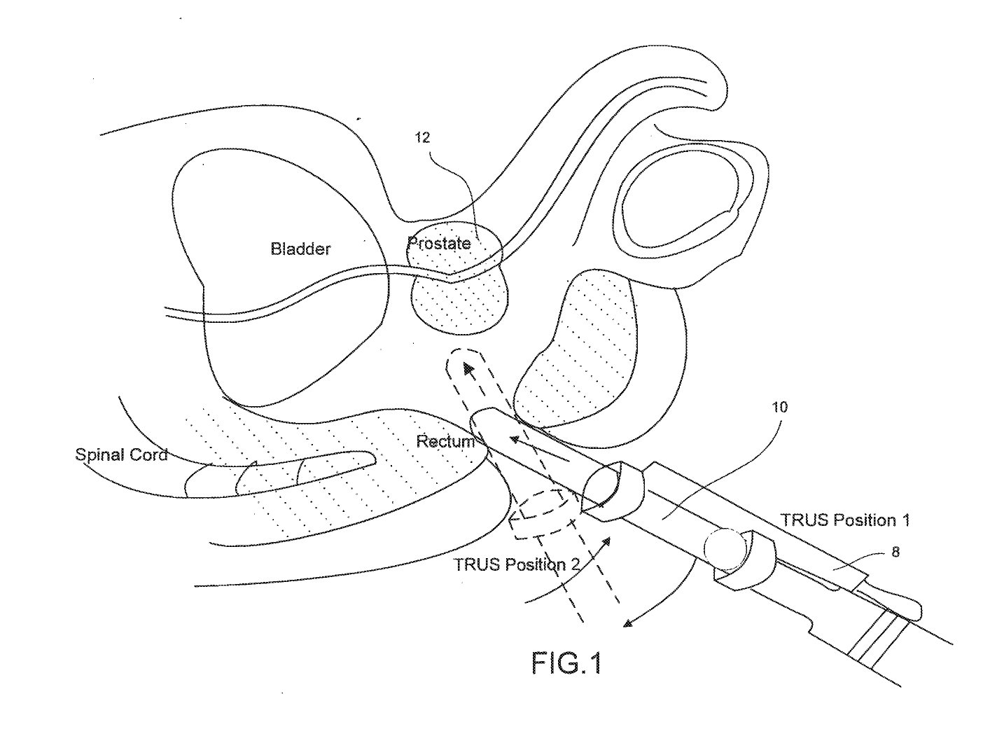

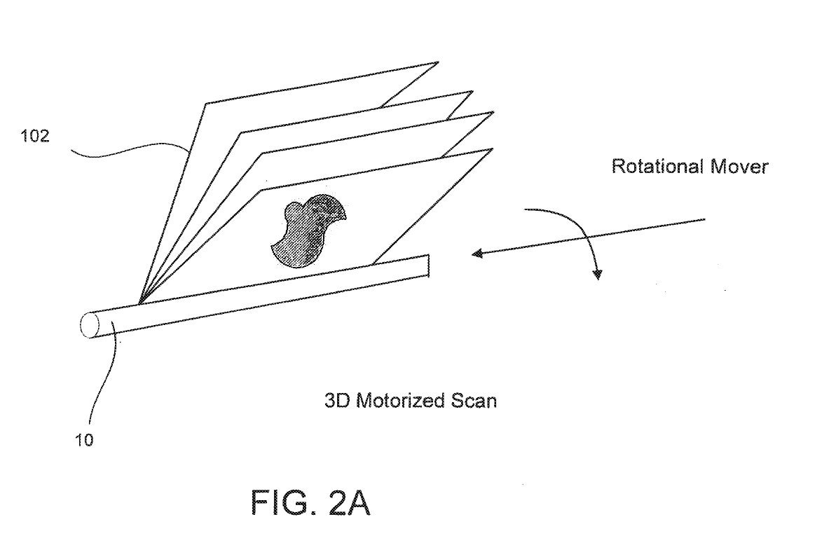

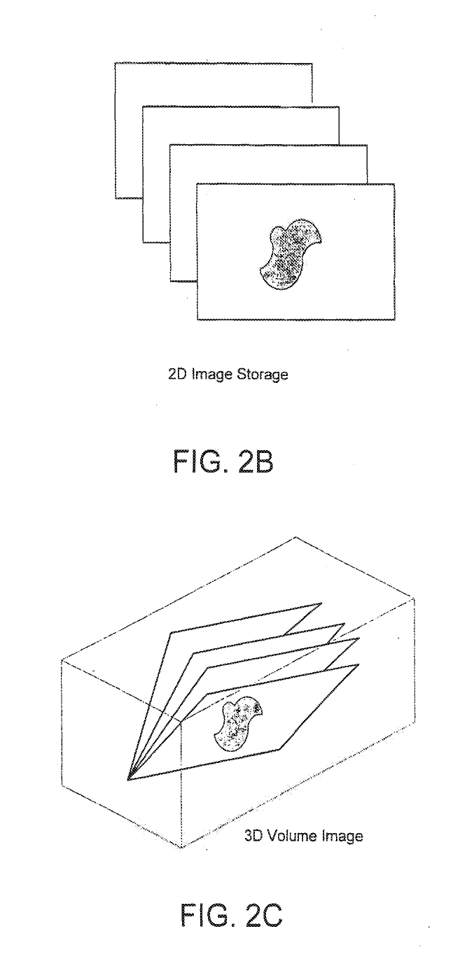

FIG. 1 illustrates a transrectal ultrasound probe that may be utilized to obtain a plurality of two-dimensional ultrasound images of the prostate 12. As shown, the probe 10 may be operative to automatically scan an area of interest. In such an arrangement, a motor may sweep the transducer (not shown) of the ultrasound probe 10 over a radial area of interest. Accordingly, the probe 10 may acquire plurality of individual images while being rotated through the area of interest (See FIGS. 2A-C). Each of these individual images may be represented as a two-dimensional image. Initially, such images may be in a polar coordinate system. In such an instance, it may be beneficial for processing to translate these images into a rectangular coordinate system. In any case, the two-dimensional images may be combined to generate a three-dimensional image (See FIG. 2C).

As shown in FIG. 2A, the ultrasound probe 10 is a side scan probe. However, it will be appreciated that an end scan probe may be utilized as well. In any arrangement, the probe 10 may also include a gun 8 that may be attached to the probe. Such a gun 8 may include a spring driven needle that is operative to obtain a core from desired area within the prostate. Alternatively, the gun 8 may plant a therapy seed at a target location within the prostate. In this regard, it may be desirable to generate an image of the prostate 12 while the probe 10 remains positioned relative to the prostate. In this regard, if there is little or no movement between acquisition of the images and generation of the 3-D image, the biopsy gun may be positioned to access an area of interest within the prostate 12.

A computer system (not shown) runs application software and computer programs which can be used to control the TRUS system components, provide user interface, and provide the features of the imaging system. The software may be originally provided on computer-readable media, such as compact disks (CDs), magnetic tape, or other mass storage medium. Alternatively, the software may be downloaded from electronic links such as a host or vendor website. The software is installed onto the computer system hard drive and/or electronic memory, and is accessed and controlled by the computer's operating system. Software updates are also electronically available on mass storage media or downloadable from the host or vendor website. The software, as provided on the computer-readable media or downloaded from electronic links, represents a computer program product usable with a programmable computer processor having computer-readable program code embodied therein. The software contains one or more programming modules, subroutines, computer links, and compilations of executable code, which perform the functions of the imaging system. The user interacts with the software via keyboard, mouse, voice recognition, and other user-interface devices (e.g., user I/O devices) connected to the computer system.

While TRUS is a relatively easy and low cost method of detecting prostate cancer and/or guiding biopsy or treatment procedures, several shortcomings may exist. For instance, some malignant cells and/or cancers may be isochoic. That is, the difference between malignant cells and healthy surrounding tissue may not be apparent or otherwise discernable in an ultrasound image. Further, speckle and shadows in ultrasound images may also make images difficult to interpret.

Other medical imaging procedures may provide significant clinical value, overcoming some of these difficulties. For example, some MRI procedures (e.g., T2-weighted MRI) may expose cancers that are isochoic, and therefore indistinguishable from normal tissue, in ultrasound imaging. MRI generally provides better soft tissue contrast than other modalities, and cancers are typically seen as lower signal intensities compared to neighboring healthy tissue. However, MRI has a disadvantage in that it is unable to distinguish other abnormalities such as benign prostatic hyperplasia or effects of therapy that also result in decreased signal intensity. MRSI imaging can overcome this limitation by revealing metabolite concentration levels, which can be used to distinguish cancer from noncancerous tissues. For example, one method is to use the ratio of concentration levels of choline and creatine with citrate, which is abnormal in the case of cancer. Despite these advantages of using MRI and MRSI to detect likely cancer locations within a prostate, ultrasound and TRUS in particular remains a more practical method for performing a biopsy or treatment procedure. Thus, it has been recognized that it would be desirable to overlay or integrate information obtained from other imaging procedures such as MRI and MRSI (i.e., a secondary image) on a TRUS image to aid in selecting locations for biopsy or treatment. However, this requires registration of the previously obtained image onto the TRUS image. For example, the secondary image may need to be rotated to align with the TRUS image. Also, because the two images are typically obtained at different times, there may be a change in shape of the prostate related to growth, patient movement or position, deformation of the prostate by the sensor probe, peristalsis, abdominal contents, etc.

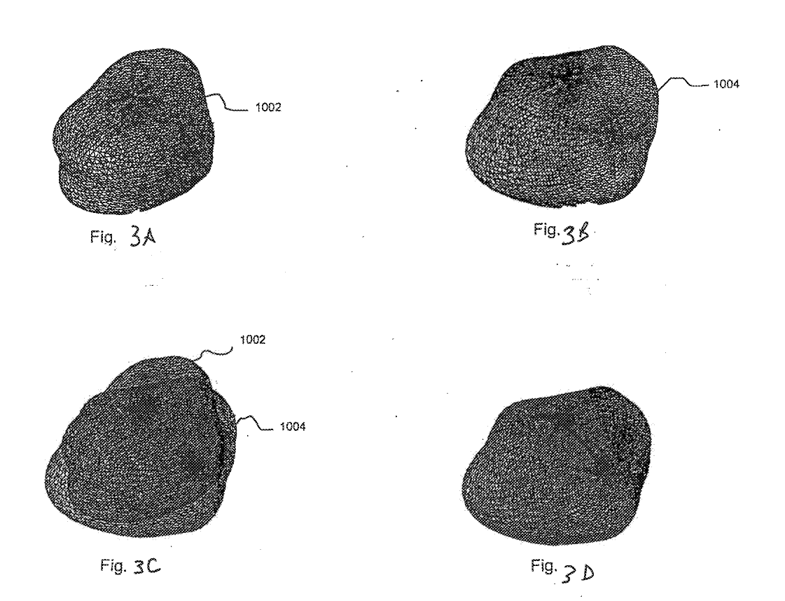

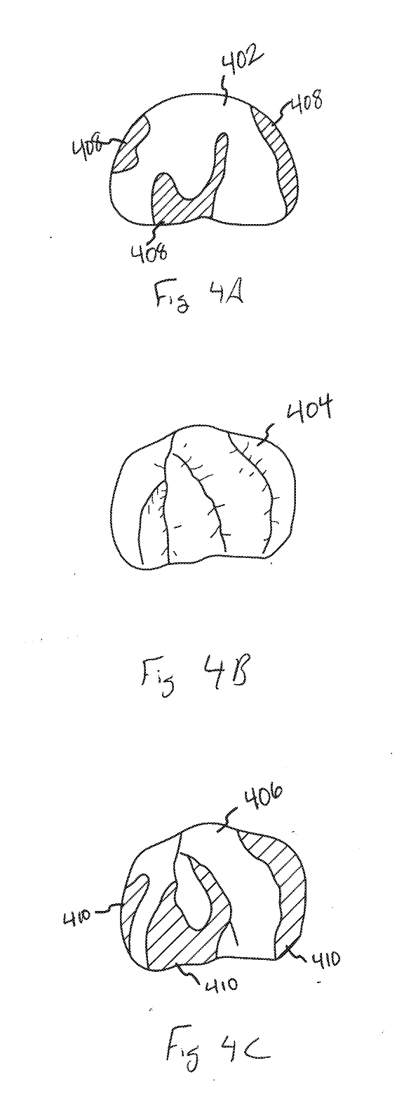

FIGS. 3A-D illustrate image registration of two prostate images obtained using different imaging modalities. In medical imaging, image registration is used to find a deformation between a pair or group of similar anatomical objects such that a point-to-point correspondence is established between the images being registered. The correspondence means that any tissue or structure identified in one image can be transferred or deformed back and forth between the two images using the deformation provided by the registration. FIGS. 3A and 3B illustrate first and second prostate images 1002 and 1004, for example, as may be rendered on an output device of physician. These images may be from a common patient and may be obtained at first and second temporally distinct times. Though similar, the images 1002, 1004 are not aligned as shown by an exemplary overlay of the images prior to registration (e.g., rigid and/or elastic registration). See FIG. 3C. In order to effectively align the images 1002, 1004 to allow transfer of data (e.g., MRI and/or MRSI data indicating likelihood of cancer) from one of the images to the other, the images must be aligned to a common reference frame and then the prior image (e.g., 1002) may be deformed to match the shape of the newly acquired image (e.g., 1004). In this regard, corresponding structures or landmarks of the images may be aligned to position the images in a common reference frame. See FIG. 3D.

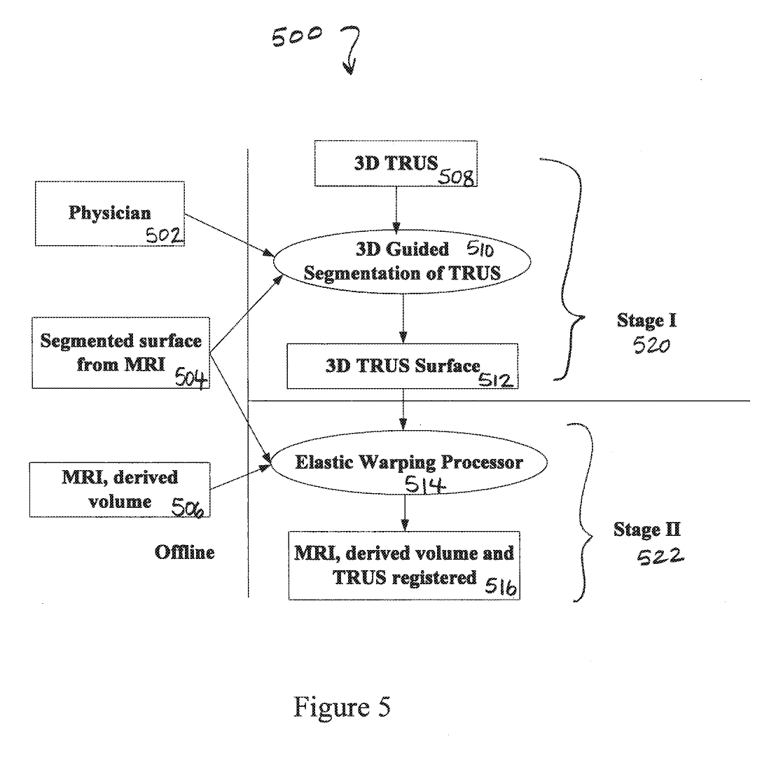

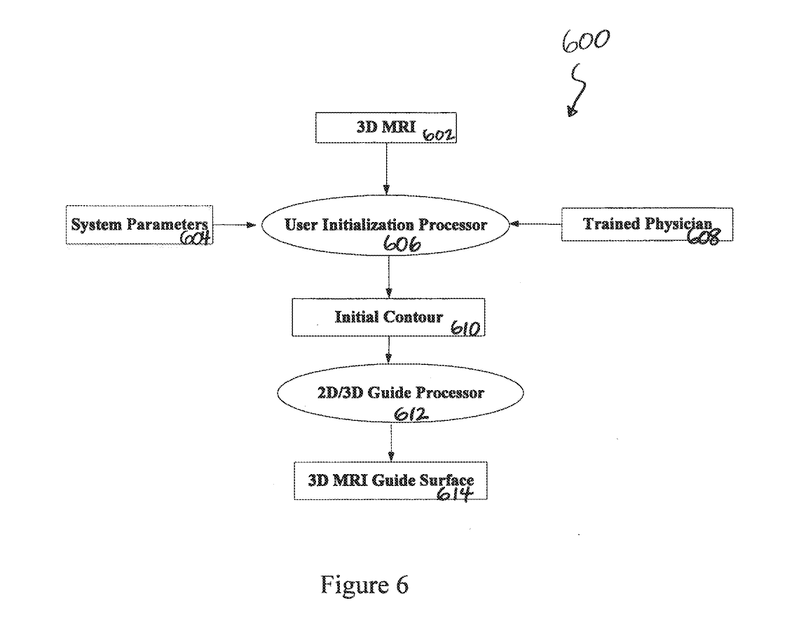

In order to quickly register a current ultrasound image with a previously obtained image (e.g., MRI/MRSI image), the current embodiment of the utility utilizes a surface registration methodology. FIGS. 4A-C and FIG. 5 diagram a system 500 for registering secondary image information, such as an MRI and/or MRSI image 402, to a TRUS image 404 to guide a biopsy or other medical procedure. Prior to performing the guided medical procedure, an MRI and/or other imaging procedure is used to obtain pertinent medical information about a prostate. In the present embodiment, an MRI/MRSI image 402 is obtained (see FIG. 4A) that includes one or more regions 408 of potentially malignant tissue. Though FIGS. 4A-C are illustrated as two-dimensional images for convenience, it will be appreciated that 3-D images may be utilized. As will be described below with reference to FIG. 6, this image may be segmented offline, e.g., before performing the TRUS procedure, to reduce the duration of the TRUS procedure and thereby minimize patient discomfort. During a first stage (520) of the guided medical procedure, a three-dimensional TRUS image 404 (see FIG. 4B) is obtained (508). A surface of the prostate is identified using any appropriate means, which may include a three-dimensional guided segmentation process (510). The TRUS segmentation process (510) may include receiving an initial boundary estimate from a physician (502) or other operator. Additionally or alternatively, segmented surface information (504) from, a previous procedure such as an MRI may be used to guide the TRUS segmentation process (510). In any case, the result is a three-dimensional TRUS surface (512) that identifies the outline of the prostate in the TRUS image.

In a second stage (522) of the guided medical procedure, an elastic warping processor (514) registers previously obtained images (506) (e.g., from MRI and/or MRSI) with the three-dimensional TRUS surface (512) to produce a multimodal image (516). See FIG. 4C. Thus, the utility may begin by obtaining MRI and MRSI data, which can be done during a common procedure prior to the TRUS-guided procedure. The MRSI data may then be processed to obtain a derived volume that indicates cancer likeliness at each voxel therein. For example, the metabolite concentrations at each voxel may be interpreted based on the levels of choline, citrate, and creatine found there, and each voxel in the image may be assigned a number that relates to how these metabolite concentrations relate to the presence or absence of malignancy. A derived image may thereby be constructed from MRI/MRSI data that is of the same size and resolution of a concurrently-obtained MRI image. The MRI and MRSI images are typically coregistered, so corresponding voxels can be directly compared. The composite image 406 (see FIG. 4C) may include tissue information from the MRI/MRSI image 402 superimposed onto and/or into the TRUS image 404 to provide a multimodal image 406.

As illustrated in FIG. 5, the process (500) may include utilizing a segmented surface from the MRI to guide segmentation of the ultrasound image. It will be noted that the MRI/MRSI image is typically obtained at a time prior to performing the ultrasound procedure. This MRI/MRSI image may be segmented prior to the ultrasound procedure to obtain a first prostate surface (e.g., a 3-D MRI surface).

This first prostate surface is used to more quickly segment the ultrasound image. In one embodiment, the system utilizes a narrow band estimation process for identifying the boundaries of a prostate from ultrasound images. As will be appreciated, ultrasound images often do not contain sharp boundaries between a structure of interest and background of the image. That is, while a structure, such as a prostate, may be visible within the image, the exact boundaries of the structure may be difficult to identify in an automated process. Accordingly, the system may utilize a narrow band estimation system that allows the specification of a limited volume of interest within an image to identify boundaries of the prostate since rendering the entire volume of the image may be too slow and/or computationally intensive, Other segmentation processes may alternatively be utilized. To allow automation of the process, the limited volume of interest and/or an initial boundary estimation for ultrasound images may be specified based on predetermined models that are based on age, ethnicity and/or other physiological criteria. That is, the initial boundary estimation may be based on previously obtained boundary information from the MRI/MRSI imaging procedure. Of course, an initial boundary estimation may be provided manually by a user.

FIG. 11 illustrates a prostate within an ultrasound image. In practice, the boundary of the prostate would not be as clearly visible as shown in FIG. 11. In order to perform a narrow band volume rendering, an initial estimate of the boundary must be provided. In one embodiment, the initial boundary estimate may be provided by stored data (e.g., previously segmented MRI data). As the stored (e.g., MRI) data is from the same prostate, use of the MRI boundary provides a good initial boundary estimate and speeds the process of segmentation. The stored data may be provided to generate an initial contour or boundary 18. Accordingly, an inner boundary 14 and an outer boundary 16 may be identified, wherein the outer boundary 16 may be provided in a spaced relationship to the inner boundary 14 and wherein the initial boundary 18 from the stored (e.g., MRI) data is contained between the inner and outer boundaries 14, 16. Accordingly, the space between these boundaries 14 and 16 may define a band (i.e., the narrow band) having a limited volume of interest in which rendering may be performed to identify the actual boundary of the prostate 12. It will be appreciated that the band between the inner boundary 14 and outer boundary 16 should be large enough such that the actual boundary 12 of the prostate lies within the band. A method for determining the actual boundary of the prostate 12 is described in U.S. patent application Ser. No. 11/615,596 titled “Object Recognition System for Medical Imaging” filed on Dec. 22, 2006, which is incorporated herein by reference. Such segmentation may be performed on a slice by slice basis to generate a 3-D surface size of the ultrasound image. Accurately segmenting the prostate is important because this boundary can be used to register other modalities to TRUS (e.g., MRI and/or MRSI), as will now be described.

FIG. 6 illustrates one process 600 for segmenting an MRI image that may subsequently be used for segmenting an ultrasound image and/or for registering the MRI image with an ultrasound image. A physician 608 may provide basic initialization input to the segmentation to generate (606) an initial contour 610 that is further processed by a guide processor 612 to generate a segmented surface 614. A typical initialization input could involve the selection of a few points that are non-coplanar along the boundary of the prostate. A coarse description of the prostate may be constructed using these points and further refined by the guide processor 612.

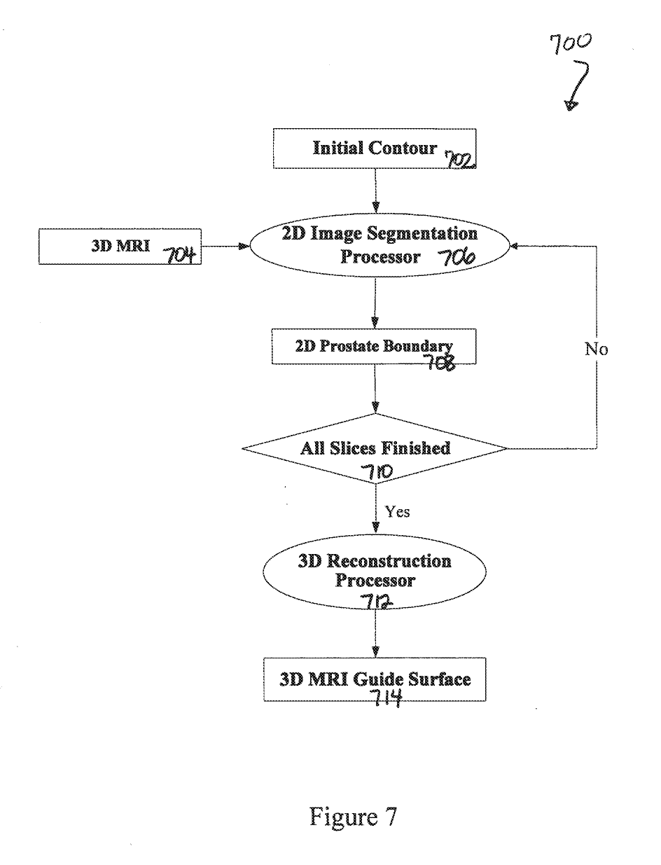

The guide processor 612 may operate on a single plane in the 3-D MRI image, i.e. refining only points that lie on this plane (2-D guide processor), or it may operate directly in 3-D using fully spatial information to allow points to move freely in three dimensions (3-D guide processor). FIG. 7 shows the general working of a 2-D guide processor 700. The initial 3-D image 704 is divided into a number of representative slices (e.g., a stack), and the boundary of the prostate may be individually computed (706) on each slice with no consideration of voxels in neighboring slices. This method is typically faster because of the reduced dimension but can also be potentially less robust due to lack of information from the third dimension. Each slice is individually segmented, in parallel or in sequence (706, 708, 710). When running in sequence, the boundaries may be allowed to propagate across neighboring slices to provide a good starting guess. After all slices are segmented (710), the points that describe the prostate are collectively used to produce (712) a triangulated mesh that hugs the prostate boundary in the 3-D MRI image 714.

Alternatively, a more sophisticated approach may allow a coarse initial description of the prostate to evolve fully in 3-D so as to result in a surface that segments the prostate in MRI. Specifically, a 3-D image segmentation processor may use several criteria in the evolution of points towards the boundary of the prostate like evolving towards high image gradients, and/or satisfying some model or smoothness criteria simultaneously.

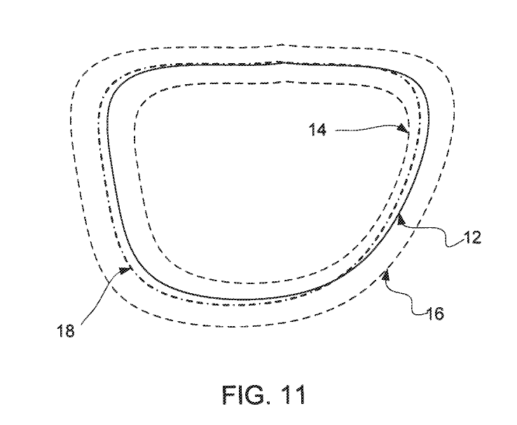

Additional information may be obtained from the MRI image prior to performing the TRUS-guided medical procedure. For example, distinguishing the transition zone of the prostate from the peripheral zone during the visualization of TRUS images could add significant clinical value. Because more than 80% of the cancers are in the peripheral zone, knowledge of its boundaries can help plan biopsy protocols more effectively. FIG. 10 shows the annotation of the transition zone 758 from a 3-D MRI image 752. This may be accomplished manually by a trained user 754 (e.g., a urologist) or with the aid of a sophisticated segmentation method such as a transition zone processor 756 capable of distinguishing regions of finer contrast that separate the transition and peripheral zones. After the MRI transition zone 758 is obtained offline, a 3-D TRUS image 760 is obtained during an ultrasound-guided medical procedure. Next, a mapping processor 762 maps the MRI transition zone 758 to the TRUS surface 760 to produce a 3-D transition zone surface 764, wherein the transition zone may be identified on the TRUS image.

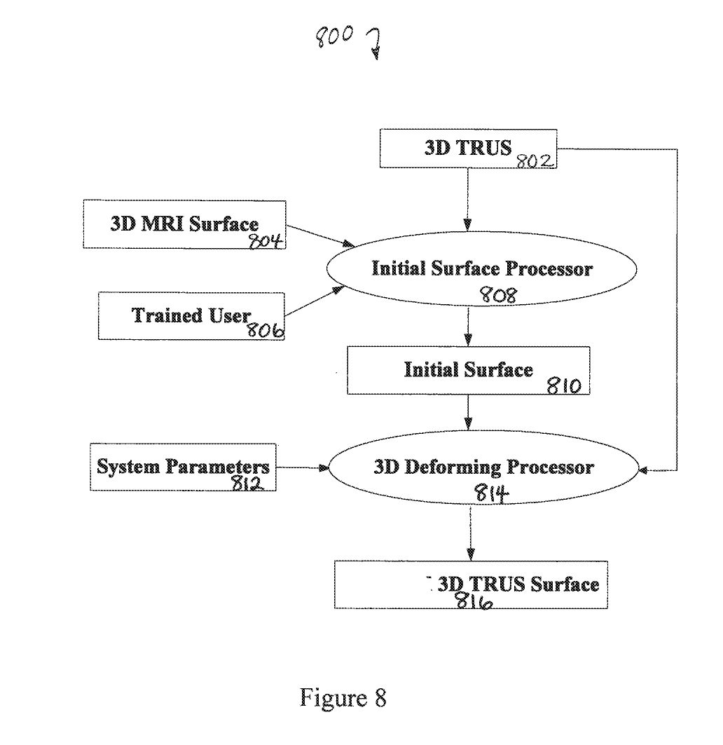

Once the supplementary (e.g., MRI) volume information has been gathered and preprocessed offline, the first stage of the TRUS-guided medical procedure may begin as described above with regard to FIG. 5. Three-dimensional segmentation of the TRUS image may be performed by a 3-D morphing processor 800 as shown in FIG. 8. An initial surface processor 808 receives 3-D TRUS data 802 from a TRUS probe. This information may be combined with user inputs 806 and/or a previously segmented 3-D MRI surface 804 as described above to produce an initial TRUS surface 810. A 3-D deforming processor 814 may then access system parameters 812 to warp the initial surface 810 into a 3-D TRUS surface 816 that follows the boundary of the prostate.

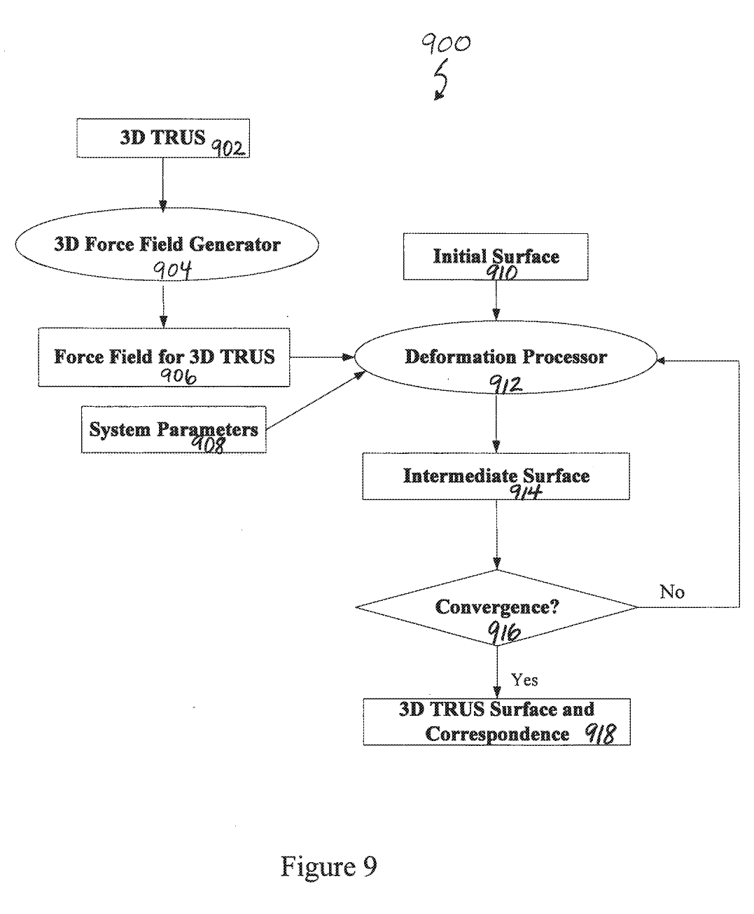

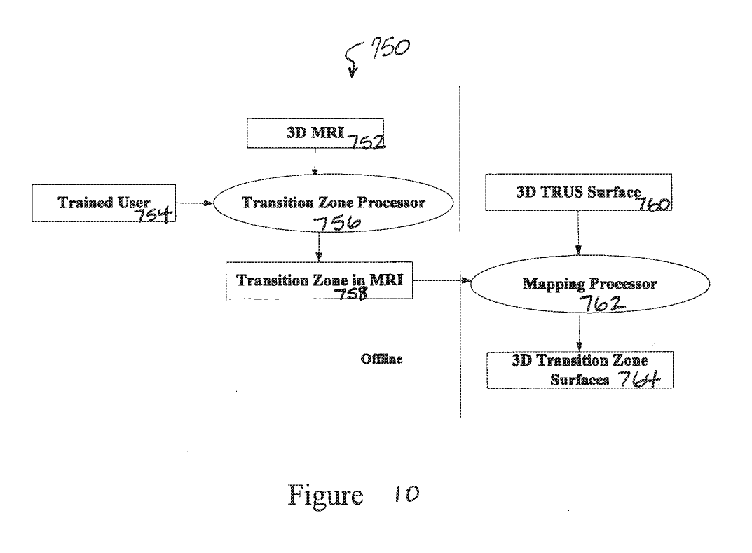

For example, FIG. 9 shows a 3-D deforming processor 900 that uses a force field estimate to deform the initial surface iteratively until the force on the surface is very small or does not change significantly (e.g., less than a set threshold). The force field may be produced, for example, by directly computing a gradient over the TRUS image or by computing gradients on a smoothed TRUS image using a low pass filter whose window width can be set appropriately. More specifically, a 3-D TRUS image is fed into a 3-D force field generator 904, which generates a force field for the TRUS image 906. The force field 906 and other system parameters 908 are combined with an initial surface 910 by a deformation processor 912 to produce an intermediate surface 914. This process is repeated until there is convergence 916 between the intermediate surface 914 and the 3-D TRUS surface 918. A similar procedure is set forth in U.S. patent application Ser. No. 11/750,854 titled “Repeat Biopsy System” filed on May 18, 2007, the entire contents of which are incorporated herein by reference.

In embodiments that use an MRI surface to segment the TRUS image, the resulting segmented TRUS surface will have the same number of vertices as those in MRI. As a result, a vertex correspondence between the two surfaces will already be available. In case the surface from TRUS has a different number of vertices from the MRI for some reason, the two surfaces will need to be explicitly registered to establish a correspondence (i.e., to relate the position of the same feature on the boundary of the prostate as seen in MRI and TRUS). The surface correspondence, once established, may be used to elastically warp the MRI and MRSI derived volumes by generating a force field on the boundary (computed from correspondences). These force fields will be allowed to propagate over the entire MRI and derived volumes displaced each voxel so as to align both MRI and derived volume to the frame of TRUS.

The TRUS operator is now provided with a multitude of information on all voxels within the 3-D volume, e.g. from the TRUS probe, structural information from MRI, and metabolite-related information from the derived volume. These volumes can be easily viewed either one at a time or in combination to improve the probability of finding cancer. See, e.g., FIG. 4C. The operator may also have a 3-D statistical cancer probability map that can be registered to the TRUS volume that can help pick target sites statistically more likely to have cancer. The multimodal image can thus be used to identify targets and/or to guide medical equipment such as a biopsy needle to desired targets during a biopsy, brachytherapy, etc.

An advantage of the surface-based registration techniques described above is their scalability with processor optimization (e.g., graphical processing unit (GPU) improvements). Images or surfaces can be split into several thousands of threads each executing independently. Data cooperation between threads is also made possible by the use of a shared memory. A GPU-compatible application programming language (API), e.g. nVidia's CUDA can be used to accomplish this task. It is generally preferable to design code that scales well with improving hardware to maximize resource usage. First the code is analyzed to see if data parallelization is possible. Otherwise algorithmic changes are suitably made so as bring about parallelization, again if this can be done. If parallelization is deemed feasible, the appropriate parameters on the GPU are set so as to maximize multiprocessor resource usage. This is done by finding the smallest data parallel thread, e.g. for vector addition, each vector component can be treated as an independent thread. This is followed by estimating the total number of threads required for the operation, and picking the appropriate thread block size that runs on each multiprocessor. For example, in CUDA selecting the size of each thread block that runs on a single multiprocessor determines the number of registers available for each thread, and the overall occupancy that can affect computation time. Other enhancements may involve, for example, coalescing memory addressing, avoiding bank conflicts, or minimizing device memory usage to further improve speed.

The strategy for GPU optimization for each of the processing steps, namely registration, segmentation, and warping, is now described. First, segmentation of a prostate from MRI or segmentation of the prostate from TRUS guided by MRI may include allowing an initial surface to evolve so as to converge to the boundary of the respective volumes. Segmentation of the MRI may be performed in two or three dimensions. In either case, points intended to describe the prostate boundary evolve to boundary locations, e.g. locations with high gradients, or other criteria. Each vertex may be treated as a single thread so that it evolves to a location with high intensity gradient. At the same time, status of neighboring vertices for each vertex can also be maintained during the evolution to adhere to certain regularization criteria required to provide smooth surfaces.

Registration of a prostate surface from MRI and TRUS may include estimating surface correspondences, if not already available, to determine anatomical correspondence along the prostate boundaries from both modalities, this may be accomplished by a surface registration method using two vertex sets, for example sets A and B belonging to MRI and TRUS, respectively. For each vertex in A, the nearest neighbor in B is found, and vice versa, to estimate the force and reverse forces acting on the respective vertices to match the corresponding set of vertices. The computations may be parallelized by allowing individual forces (forward and reverse) on each vertex to be computed independently. The forward force computations are parallelized by creating as many threads as there are vertices in A, and performing a nearest neighbor search. For example, a surface A having 1297 vertices could run as 40 threads/block containing 33 blocks. The threads corresponding to vertices beyond 1297 would not run any tasks. A similar procedure may be applied to compute the reverse force. Once forces are estimated, smoothness criteria may be similarly enforced as described in the segmentation step by maintaining the status of neighboring vertices for each vertex.

Finally, elastic interpolation of MRI and/or derived volume data to register with TRUS may include estimating the surface correspondence of the prostate from MRI to TRUS, after which the MRI and derived volumes may be elastically interpolated using these (surface correspondence) boundary conditions so as to deform the MRI and derived volumes on to the TRUS image. The 3-D volume grids corresponding to MRI and the derived volume may be subdivided into numerous sub-blocks and iteratively solved so that nodes within the 3-D volume at boundary locations deform exactly while other nodes deform as per the differential equation governing an elastic material. Each of the sub-blocks may run on a single processor. The interpolation may be performed iteratively using parallel relaxation, wherein node positions for all nodes in the 3-D volume are updated after each iteration.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A method for use in medical imaging of a prostate of a patient, comprising:
obtaining first surface information from first volume data of a prostate of a patient obtained using a magnetic resonance imaging procedure;
obtaining an ultrasound volume of the prostate of the patient using ultrasound;
segmenting the ultrasound volume to produce ultrasound surface information;
registering the first volume data to the ultrasound volume using the first surface information and the ultrasound surface information;
generating a multimodal image wherein the first volume data is displayed in a frame of reference of the ultrasound volume; and
using the multimodal image to guide a medical procedure.

2. The method of claim 1, further comprising:
obtaining second volume data of the prostate of the patient using a magnetic resonance spectroscopy imaging procedure, wherein the second volume data is co-registered with the first volume data;
extracting a derived volume from the second volume data, wherein the derived volume includes information about cancer prevalence; and
using the derived volume to guide the medical procedure.

3. The method of claim 1, wherein the medical procedure includes at least one of biopsy, brachytherapy, and targeted focal therapy.

4. The method of claim 1, wherein segmenting the ultrasound volume to produce ultrasound surface information includes using the first surface information to provide an initialized surface.

5. The method of claim 4, wherein vertices on the initialized surface evolve in two dimensions.

6. The method of claim 4, wherein vertices on the initialized surface evolve in three dimensions.

7. The method of claim 6, wherein first vertices belonging to a first slice provide initialization inputs to second vertices belonging to a second slice adjacent to the first slice.

8. The method of claim 1, wherein registering the first volume data to the ultrasound volume comprises:
establishing a surface correspondence between the first surface information and the ultrasound surface information; and
deforming the first surface information to match a boundary on the ultrasound surface information.

9. The method of claim 1, wherein registering the first volume data to the ultrasound volume includes warping the first volume data to the ultrasound volume using a nonlinear interpolant that employs surface correspondences for warping.

10. The method of claim 1, further comprising:
registering a statistical atlas to the ultrasound volume; and
using the statistical atlas to guide the medical procedure.

11. The method of claim 1, wherein obtaining first surface information from first volume data includes accessing stored MRI data.

12. A method for use in medical imaging of a prostate of a patient, comprising:
obtaining segmented MRI surface information for a prostate;
performing an MRSI procedure on the prostate to obtain a cancer indicator at each of a plurality of voxels;
extracting a derived volume from the cancer indicators;
performing a TRUS procedure on the prostate of the patient, wherein the segmented MRI surface information is used to identify a three-dimensional TRUS surface;
elastically warping the segmented surface information and the derived volume onto the three-dimensional TRUS surface to obtain a multimodal image of the prostate; and
guiding a medical procedure using information from the multimodal image.

13. The method of claim 12, wherein the step of elastically warping is performed in real time during the TRUS procedure.

14. The method of claim 12, wherein identifying a three-dimensional TRUS surface includes using a force field estimate to deform an initial surface.

15. The method of claim 12, wherein elastically warping the segmented surface information and the derived volume onto the three-dimensional TRUS surface includes:
generating a force filed on a boundary of the segmented surface information; and
propagating the force filed through the derived volume to displace a plurality of voxels.

16. The method of claim 12, wherein the step of elastically warping is performed in two dimensions on a slice-by-slice basis.

17. The method of claim 12, wherein the step of elastically warping is performed in three dimensions.
